(19) United States
(12) Patent Application Publication
(10) Pub. No.: US 2003/0134878 A1
(43) Pub. Date: Jul. 17, 2003

(54) Use of a compound in providing refreshedness on waking and a method for the treatment of drowsiness therewith

(76) Inventor: Palanlswamy Sunderraj, Stelnmur (CH)

Correspondence Address: NIXON & VANDERHYE, PC, 1100 N GLEBE ROAD, 8TH FLOOR, ARLINGTON, VA, 22201-4714, US

(21) Appl. No.: 10/305,354

(22) Filed: Nov. 27, 2002

(30) Foreign Application Priority Data
Nov. 30, 2001 (GB) 0128674.9

(51) Int. Cl. A61K 31/4439

(52) U.S. Cl. 514/343

(57) ABSTRACT

There is disclosed the use of triprolidine for enabling an individual to wake refreshed after sleep and the method of treating such an individual with triprolidine. The triprolidine is administered shortly before a person wishes to fall asleep, preferably orally and most commonly in the form of a tablet containing less than 5 mg, eg 0.1 mg, 1.25 mg or 2.5 mg, of the active ingredient. The triprolidine is also effective in enabling an individual to sleep more easily.

The present invention relates to a method for the treatment or prevention of grogginess, drowsiness or lethargy on waking from sleep, to the use of triprolidine as an aid to waking refreshed and to the use of triprolidine as both a sleep aid and a means to wake refreshed thereafter.

Although much is known about the use of various pharmaceutical sleeping formulations as aids to sleeping, little has been published about the possibility of a sleep aid enabling an individual to wake refreshed as opposed to merely experiencing degrees of hangover effects such as grogginess, drowsiness, lethargy, etc.

Many people experience, either on an occasional or chronic basis, difficulty in achieving a satisfactory amount of sleep. Such a problem may be attributable to external factors, such as factors causing stress or anxiety, to excessive use or misuse of stimulants (such as caffeine) or depressants (eg alcohol), or to temporary disturbance of the person's lifestyle, eg occasioned by shift-working or long-haul travel through different timezones. Difficulty in sleeping may also be caused by chronic pain, eg pain caused by sciatica etc. Whatever the cause, the condition may be generally considered to be a sleep disorder and may commonly be referred to as “insomnia”. It may manifest as difficulty in falling asleep and/or wakefulness during the desired period of sleep, leading to a shortened duration of sleep and/or disruption of the normal pattern of sleep.

The result of these difficulties will commonly be fatigue during the period of wakefulness, which may itself lead to stress and exacerbate the problem.

Various products are available to assist a user in overcoming problems of the type described above. Such products, commonly called “sleeping pills” may, however, suffer from disadvantageous side-effects. For example, while the products may be effective in sending a user to sleep, their effect may be of short duration, resulting in premature wakening. In other cases, the user may achieve the desired length of sleep but may awake with feelings of grogginess (a “hangover” effect). Such products may also be addictive.

In other circumstances, a person may not suffer from sleep disorders as such, but may simply wish to achieve a particularly good night's sleep. In other words, the use of such products may be elective, rather than necessitated by a clinical need.

In addition to this well documented problem, many people also experience difficulties on waking such as grogginess, lethargy and drowsiness; difficulty in becoming fully alert and an absence of feeling refreshed. These phenomena are not necessarily linked to the number of hours sleep or always encountered as a result of drugs taken prior to sleep such as alcohol, medication, etc. Furthermore, individuals encountering tiredness during waking hours and other individuals having difficulty with insomnia resort to sleep aids in an attempt to increase or improve sleeptime rest. Nevertheless, it is also well documented that a negative side effect of sleep aids can also be an increased feeling of grogginess on waking.

Triprolidine, (E)-2-[1-(4-methylphenyl-3-(1-pyrrolidinyl)-1-propenyl]pyridine, is a first generation anti-histamine and has been marketed alone and, in combination with pseudoephedrine (a decongestant), for the treatment of allergic rhinitis. Triprolidine is known to have sedative effects and has been shown to have an adverse effect on the cognitive functions of users. These are undesirable side-effects for an anti-histamine and may account for the limited extent to which triprolidine has been used in clinical practice. More recently-developed, second generation anti-histamines are less prone to such side effects, and most recent studies involving triprolidine have used that compound as a positive control against which the more modem anti-histamine compounds have been compared. Such studies have generally been conducted using healthy volunteers, rather than persons suffering from any form of sleep disorder, and have been concerned with the effects of the drug on day-time performance.

One study is known to have investigated the effect of triprolidine (amongst other anti-histamines) on sleep directly (Nicolson et al, Neuropharmacology (1985) 24 3, 245-250). In that study single doses of triprolidine (10 mg or 20 mg sustained release) were given at bedtime to volunteers. It was found that triprolidine did not significantly alter “sleep onset latency” (ie the time required to fall asleep) compared with placebo. It was also found that, compared with placebo, triprolidine had no effect on wakefulness during sleep or total sleep time.

It has now been found that triprolidine surprisingly increases the level of refreshedness felt upon waking if taken before sleeping. Advantageously, this effect is observed whilst triprolidine also acts as a sleep aid in facilitating the onset of stage I sleep and whilst enhancing sleep.

The increased level of refreshedness felt upon waking after taking triprolidine prior to sleeping was not expected and there has been no known disclosure of such an effect previously encountered.

According to a first aspect of the present invention there is provided the use of triprolidine or a salt or hydrate thereof as active ingredient of an aid to waking refreshed after sleeping.

According to a second aspect of the present invention there is provided the use of triprolidine or a salt or hydrate thereof as active ingredient in the preparation of a composition for enabling an individual to wake refreshed after sleeping.

According to a third aspect of the present invention there is provided the use of triprolidine or a salt or hydrate thereof as active ingredient in the preparation of a medicament for enabling an individual to wake refreshed after sleeping.

According to a fourth aspect of the present invention there is provided the use of triprolidine or a salt or hydrate thereof in the preparation of a sleep aid which also enables an individual to wake refreshed after sleeping.

According to a fifth aspect of the present invention there is provided the use of triprolidine or a salt or hydrate thereof as active ingredient of a sleep aid which also enables an individual to wake refreshed after sleeping.

According to a sixth aspect of the present invention there is provided the use of triprolidine or a salt or hydrate thereof as active ingredient in the preparation of a medicament for the treatment or prevention of a sleep disorder which also enables an individual to wake refreshed after sleeping.

According to a seventh aspect of the present invention there is provided a method for the treatment or prevention of grogginess, drowsiness or lethargy on waking from sleep in a mammal comprising the administration to the mammal in need thereof of a non-toxic effective dose of triprolidine or a salt or hydrate thereof prior to the desired sleeping time.

According to an eighth aspect of the present invention there is provided a method for enabling an individual to wake refreshed after sleeping comprising the administration to the individual in need thereof and prior to the desired sleeping time of a non-toxic effective dose of triprolidine or a salt or hydrate thereof.

According to a ninth aspect of the present invention there is provided a method for aiding an individual's sleep and for also enabling the individual to subsequently wake refreshed after sleeping comprising the administration to the individual in need thereof and prior to the desired sleeping time of a non-toxic effective dose of triprolidine or a salt or hydrate thereof.

According to a tenth aspect of the present invention there is provided a waking refreshed aid comprising triprolidine or a salt or hydrate thereof as active ingredient in association with a pharmaceutically acceptable carrier therefor and instructions for administration thereof at or just before the desired sleeping time.

According to a eleventh aspect of the present invention there is provided a pharmaceutical formulation for the treatment or prevention of grogginess, drowsiness or lethargy on waking after sleeping, comprising triprolidine or a salt or hydrate thereof as active ingredient in association with a pharmaceutically acceptable carrier therefor and instructions for administration thereof at or just before the desired sleeping time.

According to a twelfth aspect of the present invention there is provided a pharmaceutical formulation for enabling an individual to wake more refreshed after sleeping, comprising triprolidine or a salt or hydrate thereof as active ingredient in association with a pharmaceutically acceptable carrier therefor and instructions for administration thereof at or just before the desired sleeping time.

Typically, the percentage of individuals who, after taking a dose of triprolidine before sleeptime, wake refreshed after sleeping is in the range 1-100%, more typically, 5-70%, most typically 10-35%. An especially typical range as aforesaid is 15-30% or even more especially 20-30%. Typically, by the terms “waking refreshed” or “wake refreshed” is meant that an individual felt at least refreshed on waking, preferably, the terms are defined as the individual felt very refreshed or refreshed in accordance with the Loughborough sleep log.

Typically, the percentage of individuals who, after taking a dose of triprolidine before sleeptime, wake refreshed after sleeping is more than 2%, more typically, more than 8% and most typically, more than 15%. An especially typical level as aforesaid is more than 18% or even more especially more than 20%.

By the term sleeping as referred to herein is meant an individual in at least Stage I sleep. By the term sleeptime as referred to herein is meant the time an individual desires to go to sleep.

Typically, the percentage of individuals who, after taking a dose of triprolidine before sleeptime, felt alert after sleeping is in the range 1-100%, more typically, 5-60%, most typically 10-30%. An especially typical range as aforesaid is 15-30% or even more especially 20-30%.

Typically, the percentage of individuals who, after taking a dose of triprolidine before sleeptime, felt alert after sleeping is more than 2%, more typically, more than 8%, most typically more than 12%. An especially typical level as aforesaid is more than 16%.

By the term felt alert is meant that an individual felt at least alert on waking. Preferably, the term is defined as the individual felt alert, very alert or extremely alert in accordance with the Karolinska 9-point scale.

Typically, the percentage of individuals who, after taking a dose of triprolidine before sleeptime, felt sleepy on waking is less than 25%, more typically, less than 20%, most typically less than 15%. An especially typical level as aforesaid is less than 14% or even more especially a mean level of less than 12%.

By the term felt sleepy is meant that an individual felt sleepy on waking. Preferably, the term is defined as the individual felt sleepy or very sleepy in accordance with points 8 or 9 of the Karolinska 9-point scale.

Preferably, in use of the present invention as defined herein, the mean subjective feeling of refreshedness after waking as, for instance, determined on a 5 point scale, eg, by the morning log of the Loughborough sleep log, is increased by at least 2%, more typically, by at least 4%, most typically, by at least 5%, as compared with an equivalent dose of placebo.

Typically, in use of the present invention as defined herein, the mean subjective feeling of refreshedness after waking as for instance, determined on a 5 point scale, eg. by the morning log of the Loughborough sleep log, is increased by between 1-20%, more typically, 1-15%, most typically 2-10% as compared with an equivalent dose of placebo.

The degree of refreshedness and quality of sleep may be determined by the “morning” log of the Loughborough sleep log with the highest degree of refreshedness or quality of sleep being represented as 1 and the lowest being represented as 5. Accordingly, the percentage increase in refreshedness or quality of sleep is measured in this context by the decrease in the mean refreshedness or quality of sleep.

Preferably, by the use of the present invention, the response of awakening very refreshed or refreshed, as determined, for instance, by the morning log of the Loughborough sleep log, is improved by at least 20%, more preferably, by at least, 30%, most preferably by at least 40%, as compared with an equivalent dose of placebo.

Typically, by the use of the present invention, the response of awakening very refreshed or refreshed, as determined, for instance, in accordance with the morning log of the Loughborough sleep log is improved by between 5% and 100%, more typically, by between 10% and 80%, most typically by between 20% and 60%, and especially 40-45% as compared with an equivalent dose of placebo.

Preferably, by the use of the present invention, the response of feeling extremely alert, very alert or alert, as determined, for instance, in accordance with the Karolinska 9-point scale, is improved by at least 2%, more preferably, by at least, 5%, most preferably by at least 10%, as compared with an equivalent dose of placebo.

Typically, by the use of the present invention, the response of feeling extremely alert, very alert or alert, as determined, for instance, in accordance with the Karolinska 9 point scale, is improved by between 1% and 40%, more typically, by between 2% and 30%, most typically by between 10% and 20%, as compared with an equivalent dose of placebo.

Preferably, by the use of the present invention, the response of feeling sleepy and needing to make some effort to stay awake or very sleepy, as determined, for instance, in accordance with points 8 and 9 of the Karolinska 9 point scale, is improved (ie. decreased) by at least 2%, more preferably, by at least, 4%, most preferably, by at least 10%, as compared with an equivalent dose of placebo.

Typically, by the use of the present invention, the response of feeling sleepy and needing to make some effort to stay awake or very sleepy, as determined, for instance, in accordance with points 8 and 9 of the Karolinska 9 point scale is improved (ie. decreased) by between 1% and 100%, more typically, by between 2% and 75%, most typically, by between 4% and 60%, as compared with an equivalent dose of placebo.

It will be understood that references herein to “triprolidine” include the compound (E)-2-[1-(4-methylphenyl-3-(1-pyrrolidinyl)-1-propenyl]pyridine as well as salts thereof that are acceptable for administration to the human body. Acid addition salts may particularly be mentioned, including the hydrobromide and hydrochloride salts. The hydrochloride salt. ie triprolidine hydrochloride, is particularly preferred for use in accordance with the invention. Solvates of triprolidine, notably hydrates, eg monohydrates, and to the extent that triprolidine may exist in polymorphic forms, all such polymorphs are within the scope of the invention.

The term “refreshed” as used herein means an individual waking refreshed or alert after a dose of triprolidine has been administered prior to sleep. In this context, the determination of whether an individual is feeling “refreshed” may be made by a subjective test. An example subjective test is measuring the degree of alertness on, for instance, the Karolinska scale or the feeling of being refreshed as determined by, for instance, the Loughbomugh sleep log. Alternatively, refreshedness may be based upon the inverse relationship between refreshedness and relative levels of sleepiness as determined by the Karolinska scale.

By the term individual as referred to herein is meant any mammal or human.

The administration of the active ingredient in accordance with the invention may be beneficial in that there is evidence that users feel more refreshed upon awakening, which is not the case with other treatments for sleep disorders, or indeed in the absence of any treatment, and do not experience grogginess or a “hangover” effect after the required number of hours sleep. This too is surprising in view of the fact that such feelings have been reported in relation to other active ingredients which have a comparable mode of action to that of triprolidine. Furthermore, there is no evidence that repeated use of the active ingredient over the course of several days leads to any loss of effect.

Although the active ingredient may be co-administered with another pharmacologically active agent, presently preferred formulations contain triprolidine as the sole active agent.

The active ingredient is preferably formulated in such a manner as to lead to non-sustained, substantially immediate release of the active ingredient, ie the formulation is preferably free of ingredients intended or effective to prolong or sustain release of the active ingredient.

Administration of the active ingredient in accordance with the invention may be by a variety of routes. However, most commonly the active ingredient will be administered orally. An alternative mode of administration may be administration to the mucous membranes of the nasal passages. Further modes of administration are transdermal (eg using transdermal patches or bandages), rectal (eg as suppositories), optical, sub-lingual and pulmonary.

For oral administration, the active ingredient may be put up in a variety of dosage forms. Most commonly, the active ingredient will be formulated and administered as a tablet or the like. However, formulation as capsules, lozenges, drinks or as a syrup (solution or suspension) may also be possible, as may other dosage forms such as oral sprays.

For nasal administration, the active ingredient may be formulated as a solution, emulsion or suspension and administered by means of a spray using a suitable delivery device. Alternatively, the active ingredient may be administered as a powder, either from a pressurised aerosol delivery device or from a so-called dry powder inhaler.

For formulation in the presently preferred form, ie as a tablet, the active ingredient will generally be combined with various excipients in a manner which is known per se. In particular, the tablet will generally comprise one or more diluents or bulking agents. A diluent may also serve as a disintegrant, or the formulation may incorporate a separate disintegrant. A lubricant may also be included to facilitate release of the formed tablets from the tabletting dies of a tablet forming machine.

Thus, according to a further aspect of the invention, there is provided a tablet for enabling an individual to wake refreshed after sleeping, which tablet comprises triprolidine as sole active ingredient in admixture with one or more diluents and/or a disintegrant, the tablet comprising more than 0.1 mg and less than 4.9 mg triprolidine.

As noted above the formulation may incorporate one diluent or bulking agent, or more than one. Formulations are preferred which contain blends of two or more diluents, one of which may also serve as a disintegrant.

Preferred materials for the diluent or bulking agents include polysaccharides and derivatives thereof, and saccharides.

Polysaccharides which may be used include starch, eg maize starch, cellulose, eg powdered cellulose and microcrystalline cellulose, water-insoluble modified starches, eg sodium carboxymethyl starch, water-insoluble cellulose derivatives, eg croscarmellose sodium (cross-linked sodium carboxymethyl cellulose), cross-linked polyvinylpyrrolidone and alginic acid.

Another preferred form of diluent is a saccharide. Suitable saccharides include, for example, sucrose, lactose, dextrose, sorbitol, mannitol, xylitol and maltodextrin. Lactose and sucrose are preferred saccharides. Lactose is especially preferred. Saccharide diluents may also be beneficial in terms of modifying the taste of the formulation.

Particularly preferred diluents are dicalcium phosphate, microcrystalline cellulose, eg the products sold as Avicel PH-101 and Avicel PH-102 (Avicel is a Trade Mark) by the FMC Corporation of Philadelphia, Pa., U.S.A., calcium carbonate and lactose.

Another preferred disintegrant is a croscarmellose sodium, for example the product sold as Ac-Di-Sol (Ac-Di-Sol is a Trade Mark) by the FMC Corporation. This product, when included in the formulation, also serves as a disintegrant.

The disintegrant has the effect of causing the tablet composition to disintegrate under the conditions found in the gastro-intestinal tract. Apart from croscarmellose sodium, examples of disintegrants include one or more of wheat starch, maize starch, potato starch, sodium starch glycolate, low-substituted hydroxypropyl cellulose, alginic acid, cross-linked polyvinylpyrrolidone and magnesium aluminium silicate. Preferred disintegrants are those which swell on the action of water thus causing the ingredients in the tablet to be pushed apart and out into the aqueous disintegration medium. The preferred disintegrant is croscarmellose sodium. The disintegrant is present at an effective disintegrating amount, for example up to 25% by weight of the composition, more preferably 1-25% w/w, further preferably 3-20% w/w and most preferably 5-15% by weight of the composition.

Particularly preferred compositions, in a particular tablet compositions, include a blend of a cellulosic diluent, a saccharlde diluent and a disintegrant. The preferred cellulosic diluent is microcrystalline cellulose, the preferred saccharide is lactose and the preferred disintegrant is croscarmellose sodium.

A preferred formulation, in particular a tablet formulation, comprises the cellulosic diluent, the saccharide diluent and the disintegrant in the ratio of 0.01-10 parts by weight of cellulosic diluent, 0.01-10 parts by weight of saccharide diluent to 1 part by weight of disintegrant. More preferably, the formulation contains 2-5 parts by weight of cellulosic diluent per part by weight of disintegrant, and 4 to 7 parts by weight of saccharide diluent per part by weight of disintegrant.

The diluents and/or disintegrant are preferably incorporated into the compositions in finely divided (powder) form.

The diluents and disintegrant preferably together constitute in excess of 80% w/w of the tablet formulation, more preferably in excess of 90% w/w, and most preferably in excess of 94% w/w.

The lubricant may be, for example, stearic acid, a metallic stearate, a polyethylene glycol of molecular weight of 4,000 or more, or purified talc. The preferred lubricant is a metallic stearate, particularly magnesium stearate, which may be present in the formulation at relatively low levels, typically less than 1% or 0.5% by weight.

It has been found to be particularly advantageous for the tablet formulation to be formed with a coating, preferably a sugar coating or film coating process, more preferably a film coating comprising a hydrophilic polymer, particularly a cellulose derivative such as a methylated cellulose derivative, eg hydroxyethylmethylcellulose and, particularly, hydroxypropylmethylcellulose.

The coating may also comprise an inorganic filler material, most preferably french chalk, to enhance the physical properties of the coating and prevent cracking etc, and also a pigment, eg a titanium dioxide pigment dispersion.

It has been found that, in addition to improving the appearance of the tablet and acting as a barrier to ingress of moisture, the film coating is also effective in masking the taste of the active ingredient.

The tablet formulation may be prepared by a process involving dry blending or wet or dry granulation. However, it is preferred to use a manufacturing method which involves direct compression into a tablet without an intermediate, eg a wet or dry granulation, stage.

The formulation may be made by dry mixing the active ingredient with the other ingredients, eg the lubricant and diluents and disintegrant, eg in a powder blending machine. It is particularly preferred that the active ingredient is dispersed by progressive dilution with agitation in a proportion, eg about one-half, of the excipients so as to achieve even distribution of the active ingredient in the excipients, and then to add the remainder of the excipients with further agitation and mixing. The mixture may then be compressed in a tablet forming machine and a coating, preferably a sugar coat or a film coat may then be applied to the tablets so formed by spraying the tablets with a solution or suspension of the coating-forming ingredients while the tablets are tumbled.

Such a direct tablet compression manufacturing method has been found to be beneficial in that it avoids problems attributable to crystal growth and changes in morphology which might occur in a wet granulation process.

Other, currently less preferred, dosage forms may be prepared in a manner which is generally known per se. For example, syrups may be prepared by dissolving or suspending the active ingredient in a liquid vehicle, eg water, optionally with suspending agents or the like, eg cellulose derivatives, gums etc.

For administration by inhalation, via nose or mouth, the formulations may be formulated with a compressed gas or liquefied gas propellant, eg any conventionally used propellant such as a chlorofluorocarbon, hydrofluorocarbon, compressed hydrocarbon, nitrogen etc. Alternatively, the active ingredient may be formulated as a dry powder, generally in admixture with a diluent such as crystalline lactose.

The amount of active ingredient to be administered in a single dose may vary quite widely, depending inter alia on the desired effect and the mode of administration. However, a formulation for oral administration, eg a tablet, will generally contain at least 0.5 mg and less than 10 mg of active ingredient, more commonly no more than 5 mg, eg 1-25 or 2.5 mg. Doses of formulations for administration by nasal and sub-lingual administration, which would be expected to deliver the active ingredient more quickly and efficiently, may contain less active ingredient, eg between 0.1 and 1-0 mg, eg about 0.5 mg and generally at a level of 20% of the oral dose levels mentioned herein. Preferably, such nasal and sub-lingual formulations contain active ingredient in the range 0.01-2.5 mg, more preferably, 005-1.0 mg and most preferably, 0.1-0.5 mg.

In general, the desired dose (which may comprise one or more unit doses, eg one or two tablets or the like) will be taken by a user prior to the desired time at which it is desired for the composition to take effect. Most commonly, the dose will be taken at night-time, ie prior to the user sleeping through hours of darkness. Typically, the dose may thus be taken after 8 pm in the evening or later, say after 9 pm or after 10 pm. Typically, it may be recommended that the user take the composition between 0, more commonly 1 minute and 2 hours prior to the time at which he or she wishes to fall asleep. Most commonly, the composition may be taken about 10 to 30 minutes prior to that time. In addition, however, the active ingredient may be effective, particularly at lower doses, in restoring sleep, eg in the event of night-time waking.

Preferably, the use of triprolidine in any aspect of the invention as defined herein is its use as active ingredient. Preferably, the triprolidine in any aspect of the invention defined herein is in the form of a non-toxic effective dose, preferably, suitable for any given mammal or human and determined in accordance with age and weight.

Preferably, to obtain the benefits on waking as defined herein, the active ingredient of triprolidine administered before sleeptime is less than 10 mg, typically less than 5 mg, more preferably, less than 4.5 mg, most preferably less than 4.0 mg. Especially preferred is a dose as aforesaid of less than 3.5 mg and most especially preferred is a dose of less than 3.0 mg. Typically, the dose of triprolidine is between 0.01 and 10.0 mg, preferably, between 0.01 and 4.9 mg, more preferably, between 0.01 and 4.5 mg, most preferably between 0.5 and 4 mg. Especially preferred is a dose of between 1 and 3.5 mg and more especially a dose of between 2.0 and 3.0 mg. Most especially preferred is a dose as aforesaid of about 2.5 mg or 1.25 mg. Preferably, the above dosage levels are based on triprolidine hydrochloride monohydrate and amounts of other salts or hydrates should be varied accordingly to deliver the equivalent amount of active ingredient.

In the formulations of the present invention, the triprolidine may be in any suitable release form such as a slow release , sustained release, immediate release or uncontrolled release form. The formulation may also be in any one or more of the following delivery forms:

Pastilles

lozenge

chewable tablets

fondant-fill tablets

coated or uncoated tablets

sublingual tablets

fast-melt tablets

hot or cold drinks

syrups

drops

emulsions

dry powder

suspension

transdermal patch

suppository.

Preferably, the dose of the triprolidine in accordance with the invention may be taken by an individual before it is desired to go to sleep (sleeptime), preferably less than two hours before sleeptime, more preferably, less than one hour before sleeptime, most preferably, less than 20 minutes before sleeptime. Especially preferred is to take the dose of triprolidine less than 15 minutes before sleeptime.

Preferably, the dose of triprolidine is less than 4 doses per day (24 hour period), more preferably, less than 3 doses per day, most preferably less than 2 doses per day. Especially, preferred is 1 dose per day.

The packaging of the invention as defined herein may be in any suitable form such as, for example, a blister pack, bottle, tamper-proof container, sachet, box, etc. The packaging of the invention may be associated with instructions for any of the features or preferred features of the invention as defined herein.

For the avoidance of doubt, reference to the “use of the present invention” herein should be taken to include “the method of the invention”, and “use of a pharmaceutical formulation” as well as use of the present invention per se.

Advantageously, the use of triprolidine in the present invention results in a reduced hangover or morning grogginess effect as compared with other sleep aids or sleep disorder remedies. More advantageously, the use of triprolidlne in the present invention provides an improved degree of refreshedness or more refreshed feeling upon waking as determined by the Loughborough sleep log or Karolinska scale and as compared with placebo.

For the avoidance of doubt, reference to quantities of triprolidine herein should be taken as references to quantities of the hydrochloride mono hydrate (HCl.H₂O) form. However, it should be appreciated that the invention extends to other forms, including all pharmaceutically active salts and hydrates thereof.

The term refreshed as used herein may be substituted by any term selected from alert, invigorated, revitalised, re-energised, recharged, rejuvenated, attentive, awake or words having the like effect or equivalent general meaning and the term refreshedness may also be substituted by the grammatical equivalent thereof from the words aforesaid. In addition, the term alert as used herein can be substituted by any of the above alternative terms.

Examples of tablet formulations which may be used in the invention are as follows:

### EXAMPLE 1

### 5 mg Tablet

| | | Parts by weight/ |
|---|---|---|
| | Ingredients | mg per tablet |
| 1 | Triprolidine | 5 |
| | hydrochloride BP | |
| 2 | Microcrystalline | 87.5 |
| | cellulose 102 | |
| 3 | Lactose | 137.5 |
| 4 | Magnesium stearate BP | 1 |
| 5 | Croscarmellose sodium | 25 |
| 6 | Opaspray White M-1-7111B | 1.08 |
| 7 | French chalk for tablets | 0.65 |
| 8 | Hydroxypropylmethylcellulose | 3.27 |
| | 2910 USP 606 | |

Method

(a) Triprolidine hydrochloride (1) was mixed with approximately one-half of the components (2)-(5) and thoroughly mixed. The remainder of components (2)-(5) were added and mixing continued to achieve uniform distribution of the active ingredient in the mixture.

(b) The mixture was compressed to form tablets, each containing 5 mg of active ingredient, in a tablet forming machine.

(c) The tablets were film-coated by spraying with an aqueous suspension of components (6)-(8) containing 15% solids while being tumbled, followed by drying.

### EXAMPLE 2

### 2.5 mg Tablet

| | | Parts by weight/ |
|---|---|---|
| | Ingredients | mg per tablet |
| 1 | Triprolidine | 2.5 |
| | hydrochloride BP | |
| 2 | Microcrystalline | 87.5 |
| | cellulose 102 | |
| 3 | Lactose | 137.5 |
| 4 | Magnesium stearate BP | 1 |
| 5 | Croscarmellose sodium | 25 |
| 6 | Opaspray White M-1-7111B | 1.08 |
| 7 | French chalk for tablets | 0.65 |
| 8 | Hydroxypropylmethylcellulose | 3.27 |
| | 2910 USP 606 | |

Method

Prepared by a method analogous to Example 1.

### EXAMPLE 3

Example 3 was produced in accordance with the following composition and constituted the trial formulation unless otherwise mentioned hereinafter. Patients received one tablet for the 2.5 mg dose and two tablets for the 5.0 mg dose.

| | Name of Ingredient | mg/tablet |
|---|---|---|
| 1. | Triprolldine HCl. H₂O | 2.5 |
| 2. | Micro-crystalline Cellulose | 29.0 |
| 3. | Lactose H₂O | 60.0 |
| 4. | Magnesium Stearate | 1.0 |
| 5. | Croscarmallose Sodium | 10.0 |

Method

Example 3 was prepared by the method analogous to example 1 (a) and (b) above.

### EXAMPLE 4

Example 4 was produced in accordance with the following composition and method and provides an example of an alternative fast melt formulation.

Triprolidine Fast Melt Tablets (2.5 mg)

| Ingredient | Functionality | % w/v |
|---|---|---|
| Triprolidine Hydrochloride | Active | 2.5 mg |
| Mannitol | Filler/sweetener | 400 mg |
| Sodium Croscarmellose | Disintegrant | 25 mg |
| Aspartame | Sweetener | 20 mg |
| Precipitated Silica | Flow aid | 10 mg |
| Flavour | Flavour | qs |
| Magnesium Stearate | Lubricant | 2.5 mg |
| Total | | 460 mg |

Blend the triprolidine, manitol, aspartame, sodium croscarmellose, silica and flavouring for 20 minutes in a suitable blender. Add the magnesium stearate and further blend for 5 mins. Compress the blend into tablets of weight 460 mg.

Examples 5-7 illustrate further formulations for the triprolidine of the present invention.

### EXAMPLE 5

Triprolidine Sugar Free Syrup (2.5 mg/5 ml)

| Ingredient | Functionality | % w/v |
|---|---|---|
| Triprolidine Hydrochloride | Active | 0.05 g |
| Purified Water | Solubilizer | 50% |
| Natrosol 250 HX | Thickener | 0.6 |
| Glycerin | Sugar free diluent | 20% |
| Lycasin 80/55 | Sugar free diluent | 20% |
| Acesulfame K | Sweetner | 0.075 |
| Domiphen Bromide | Preservative | 0.01 |
| Flavour | Flavour | qs |
| Colour | Colour | qs |
| Purified Water to | | 100% |

Dissolve the triprolidine in purified water in a suitable vessel. Stir until a clear solution is produced. In a separate vessel add the glycerin and the lycasin, heat to 40° C. Slowly add the Natrosol. Recirculate through an in-line Sliverson ® with a 2 mm screen until all the lumps have disappeared and the bulk is uniform.

Add the Natrosol solution to the triprolidine solution via the in-line Silverson ®. Add with stirring the Domiphen Bromide, Acesulfame K, flavour and Colour. Stir until a homogenous mix is produced and pass through a 60 mesh sieve into bulk containers.

### EXAMPLE 6

Triprolidine Hot Drink (2.5 mg/sachet)

| Ingredient | Functionality | mg/sachet |
|---|---|---|
| Triprolidine Hydrochloride | Active | 2.5 |
| Acesulfame Pottasium | Sweetner | 12.5 |
| Aspartame | Sweetner | 12.5 |
| Malted milk Flavour | Flavour | 200 |
| French Vanilla Flavour | Flavour | 225 |
| Lactose | Filler | 2547.5 |
| Purified Water | Granulating solution | qs |
| Total | | 3000 mg |

The triprolidine is dissolved in purified water. Lactose, aspartame and acesulfame are sieved and dry mixed before being granulated with the previously prepared triprolidine solution. The granules are fluid bed dried, sieved and blended with the flavours.

### EXAMPLE 7

Triprolidine Pastille (2.5 mg)

| Ingredient | Functionality | mg/pastille |
|---|---|---|
| Triprolidine hydrochloride | Active | 2.5 |
| Gum Arabic | Natural gum | 986 |
| Maltitol syrup | sugar free diluent | 859.5 |
| Glycerin | sugar free diluent | 81 |
| Citric Acid | pH adjuster/flavour enhancer | 39 |
| Flavour | Flavour | 23 |
| Acesulfame K | Sweetner | 2 |
| Hibiscus Extract | Flavour | 4 |
| Miglyol Oil - 866 | surfactant | 4 |
| Water | | 299 |
| Total | | 2300 mg |

The gum is dispersed in water (95° C.), with stirring. Maltitol syrup and glycerin are mixed and pumped in to the pre-cooker at 126° C. The gum solution is pumped into the maltitol syrup solution and mixed. The triprolidine, flavours and colours are added to the mixture.

The pastille mixture is pumped from the dispenser to the depositing hopper to form the pastilles in the starch mould boards. The pastilles are left to gel for 6-8 days.

Clinical Trial

The efficacy of triprolidine in enabling a patient to feel refreshed or alert upon waking after taking triprolidine prior to sleeptime was investigated using patients with a history of sleep disorders and utilising triprolidine prepared in accordance with example 3.

The studies herein utilised the following determination methods:

(a) Karolinska scale as defined in: Int. J. Neuroscience 52 29-37 (1990); and validation: Sleep 17 (3) 236-41 (1994)

(b) Loughborough Sleep log as defined in : Sleep 17 (2) 146-159 (1994); and Sleep 19 (2) 127-134 (1995)

(c) Actimetry—AW4 actimeters (Cambridge Neurotechnology) were worn continuously throughout the study. A button was pressed at night when the subject desired to go to sleep and again in the morning upon waking. The results of the actimeter study were analysed in the manner defined by Home et al (Sleep, 17(2); 146-159).

SDI% was calculated as follows:

$$SDI = \frac{\text{Number of 30 second epochs with movement}}{\begin{matrix}\text{Number of 30 second epochs} \\ \text{from total time spent in bed}\end{matrix}} \times 100$$

This is the measure of:

1. The length of time it took to fall asleep

2. Any awakenings throughout the sleep period

Expressed as a % of total time spent in bed.

Study Objectives

To evaluate the effects of two doses of triprolidine compared with placebo.

Study Design

A multiple-dose, placebo-controlled, parallel-group, double-blind, randomised study investigating the effects of 2.5 mg and 5 mg triprolidine in patients with temporary sleep disturbance.

Male and Female candidates aged 18 years and above were recruited to one of five research centres by means of local advertising. Candidates were screened by means of a telephone questionnaire and selected candidates invited for interview at the research centre. Key inclusion criteria used to select candidates for the study were:

A record of poor sleep at least 2 nights per week

A record of poor sleep for at least 1 week but not more than 3 months

Sleep disturbance not caused by underlying disease

No excess use of alcohol or drugs

Sleep disturbance affected daytime functioning

The candidates came to the research centre on Thursday or Friday and were fitted with a wrist actimeter (AW4 from Cambridge Technology) to establish a baseline measure for SDI and were provided with diary cards to record subjective assessments for the Loughborough Sleep Log and the Karolinska Sleepiness Scale. They returned to the investigational site on the Monday and were issued with the study compositions (2.5 mg triprolidine, 5 mg triprolidine or placebo). The investigator telephoned a central randomisation centre where the subject was randomised to a particular treatment group using a dynamic balanced randomisation algorithm. The subject was given three doses of their allocated study medication and instructed to take a single dose of two tablets 20 minutes before they intended to go to sleep on three consecutive evenings. commencing that evening. The diary cards for the Loughborough Sleep Log and Karolinska Sleepiness Scale were asked to be completed on waking.

The candidates returned to the research centre on the following Friday.

Parameters Evaluated

Candidates were required to complete a questionnaire 15 minutes after awaking on the feeling of refreshedness assessed on a 5-point scale, the Loughborough sleep log.

A daytime sleepiness assessment was also made 20 minutes, 2 hours and 4 hours after awaking on the Karolinska 9-point scale, ie. the sleepiness scale.

Results

198 candidates completed the study, of whom 178 provided evaluable data. (61 placebo, 60 on 2.5 mg triprolidine and 57 on 5 mg triprolidine. The subjects on 2.5 mg dose took one tablet and those on 5 mg dose took 2×5 mg tablets. The subjects on placebo took a dose to match the active treatments.

Key results were as follows:

There was evidence that there was a lack of daytime sleepiness associated with those patients who took either dose of triprolidine

The following results were obtained for patients taking 2.5 mg triprolidine:

15 minutes after waking, patients taking triprolidine recorded feeling more refreshed than those on placebo, as determined by the Loughborough sleep log(p<0.05).

There were a greater percentage of people on 2.5 mg triprolidine who, on waking were feeling alert, very alert or extremely alert than those on placebo as measured by the Karolinska log.

There was a lower percentage of people on 2.5 mg triprolidine who, on waking were feeling sleepy, and needing to make some effort or very sleepy, needing to make a great effort to keep awake than those on placebo as measured by the Karolinska log.

There was no evidence of residual hangover effects / morning grogginess from the drug.

Further analyses show the advantageous effects of triprolidine in relation to the degree of refreshedness on waking.

The study design used 3 groups. On average, the number of individuals in each of the 3 groups (placebo, 2.5 mg triprolidine and 5 mg triprolidine) was 60±10 patients.

In the trial, patients were tested during a seven day period and the results have been analysed for a mean of three days in the middle of this period. The effects of triprolidine at dose level 25 mg and 5.0 mg are compared with placebo in table 1.

TABLE 1
Datasets (a) and (b) - Main Analyses

| | | Mean | | |
|---|---|---|---|---|
| | | Placebo | 2.5 mg | 5 mg |
| (a) 15 mins after awaking | | | | |
| (1-very refreshed | Mon | 3.41 | 3.33 | 3.72 |
| 5-very tired) | Tues | 3.46 | 3.23 | 3.56 |
| (Loughborough sleep log) | Wed | 3.42 | 3.18 | 3.54 |
| | Mean of 3 | 3.45 | 3.24 | 3.59 |
| (b) last night I slept | | | | |
| 1-extremely well, | Mon | 3.2 | 2.67 | 2.49 |
| 5-extremely badly) | Tues | 3.06 | 2.71 | 2.93 |
| (Loughborough sleep log) | Wed | 3.02 | 2.81 | 2.64 |
| | Mean of 3 | 3.11 | 2.73 | 2.69 |

Statistical Analysis

Generally the treatment groups were well balanced in terms of the demographic data Unless otherwise mentioned all group data was analysed using ANOVA. In two cases, namely, how the patient felt 15 minutes after awakening in the Loughborough Sleep Log and the Karolinska Sleepiness Scale at 20 minutes, the two variables were analysed using ANCOVA by including the weekend and the mean of Friday/Saturday/Sunday night as a covariate. The method was a closed test procedure (Williams test). Each of the tests were to be conducted at the 5% level. The analysis of the secondary endpoints was similarly conducted using the Student's t-tests on parameter estimates taken from the analysis of variance model presented above.

The following is a copy of the “Loughborough sleep log questionnaire” which was used by patients in the study and provided the data for datasets a and b in table 1.

“Loughborough Sleep Log” Questionnaire

This will be completed 15 minutes after waking.
Bedtime Log

| | | |
|---|---|---|
| I went to bed at: | | I turned out the lights at: |
| The windows are: | shut | |
| | not shut | |

Morning Log

| I woke up at this morning | I got out of bed at this morning |
|---|---|
| 15 minutes after waking I felt: | Last night I slept: |
| a) very refreshed | a) extremely well |
| b) refreshed | b) very well |
| c) neither refreshed nor tired. | c) fairly well |
| d) tired | d) rather badly |
| e) very tired | e) extremely badly |

Night Diary

| During the night the windows were left: | opened |
|---|---|
| | shut |
| During the night the secondary glazing was left: | opened |
| | shut |
| During the night my partner slept in: | the same bed as me |
| | a different bed to me |

As far as I can remember, it took me minutes to fall asleep last night
As far as I can remember, I woke up times last night
Please note the details of any awakenings you can remember in the table below.

| | | |
|---|---|---|
| Time | Length of time awake (mins) | Reason for awakening.” |

Table 2 shows additional data in connection with data set (a) showing the improvement in refreshed responses at the 2.5 mg dosage of triprolidine hydrochloride monohydrate.

TABLE 2
Loughborough Sleep Log: Awoke Very
Refreshed or Refreshed Responses

| | Day of Testing | | | | | |
|---|---|---|---|---|---|---|
| | Monday | | Tuesday | | Wednesday | |
| Dose | N | % | n | % | n | % |
| Placebo | 10 | 15.2 | 10 | 16.4 | 11 | 18.3 |
| 2.5 mg TRP.HCl.H₂O | 14 | 23 | 14 | 23 | 16 | 25.8 |
| 5 mg TRP.HCl.H₂O | 7 | 11.5 | 5 | 8.2 | 9 | 14.8 |

Similarly, table 3 shows corresponding additional data in connection with data set (b).

TABLE 3
Loughborough Sleep Log: Last Night I Slept
Extremely Well or Very Well Responses

| | Day of Testing | | | | | |
|---|---|---|---|---|---|---|
| | Monday | | Tuesday | | Wednesday | |
| Dose | N | % | n | % | n | % |
| Placebo | 11 | 18 | 12 | 22.2 | 13 | 24.1 |
| 2.5 mg TRP.HCl.H₂O | 24 | 41.4 | 23 | 41.8 | 22 | 37.9 |
| 5 mg TRP.HCl.H₂O | 30 | 50.9 | 17 | 28.8 | 24 | 39.3 |

Karolinska's sleepiness scale is set out below and the results for placebo, 2.5 and 5.0 mg doses of triprolidine are shown in tables 4 and 5. Table 4 relates to the number of individuals experiencing scales 1, 2 or 3 on the Karolinska scale and table 5 relates to the number of individuals experiencing scales 8 and 9.

Karolinska Sleepiness Scale

This will be completed 20 minutes after awakening and then at 2 hours and 4 hours following the first assessment on days 5, 6, 7 and 8.

1. Extremely alert

2. Very alert

3. Alert

4. Rather alert

5. Neither sleepy or alert

6. Some signs of sleepiness

7. Sleepy but no effort to keep awake

8. Sleepy, some effort to keep awake

9. Very sleepy, Great effort to stay awake, fighting sleep

TABLE 4
Karolinska 9-point scale (a) I feel
extremely alert, very alert or alert

| | Day of Testing | | | | | |
|---|---|---|---|---|---|---|
| | Monday | | Tuesday | | Wednesday | |
| Dose | n | % | n | % | n | % |
| Placebo | 9 | 13.6 | 14 | 230 | 11 | 17.2 |
| 2.5 mg TRP.HCl.H₂O | 13 | 21.3 | 13 | 21.3 | 13 | 21.0 |
| 5 mg TRP.HCl.H₂O | 4 | 6.3 | 6 | 9.5 | 11 | 17.5 |

TABLE 5
(b) I feel (I) sleepy, [and need to make] some effort
or (II) very sleepy, a great effort to keep awake

| | Day of Testing | | | | | |
|---|---|---|---|---|---|---|
| | Monday | | Tuesday | | Wednesday | |
| Dose | n | % | n | % | n | % |
| Placebo | 8 | 12.1 | 10 | 16.4 | 9 | 14.1 |
| 2.5 mg TRP.HCl.H₂O | 7 | 11.5 | 8 | 13.1 | 4 | 6.5 |
| 5 mg TRP.HCl.H₂O | 8 | 12.5 | 11 | 17.5 | 8 | 12.7 |

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A method for the treatment or prevention of grogginess, drowsiness or lethargy on waking from sleep in a mammal comprising the administration to the mammal in need thereof of a non-toxic effective dose of triprolidine or a salt or hydrate thereof prior to the desired sleeping time.

2. A method for enabling an individual to wake refreshed after sleeping comprising the administration to the individual in need thereof and prior to the desired sleeping time of a non-toxic effective dose of triprolidine or a salt or hydrate thereof.

3. A method for aiding an individual's sleep and for also enabling the individual to subsequently wake refreshed after sleeping comprising the administration to the individual in need thereof and prior to the desired sleeping time of a non-toxic effective dose of triprolidine or a salt or hydrate thereof.

4. The method as claimed in claim 1, wherein the dose of active ingredient of triprolidine administered is between 0.01 and 4.9 mg.

5. The method as claimed in claim 2, wherein the dose of active ingredient of triprolidine administered is between 0.01 and 4.9 mg.

6. The method as claimed in claim 3, wherein the dose of active ingredient of triprolidine administered is between 0.01 and 4.9 mg.

7. The method as claimed in claim 2, wherein the dose of active ingredient of triprolidine administered is less than 5 mg.

8. A method as claimed in claim 2, wherein the triprolidine is in the form of triprolidine hydrochloride.

9. A method as claimed in claim 2, wherein the person is suffering from a sleep disorder.

10. A method as claimed in claim 2, wherein the person is not suffering from a sleep disorder but is desirous of achieving a feeling of waking refreshed upon waking.

11. A method as claimed in claim 2. wherein the active ingredient is administered orally, nasally, optically, rectally, pulmonarily, transdermally or sub-lingually.

12. A method as claimed in claim 2, wherein the active ingredient is administered in the form of a tablet, capsule, drink, lozenge, drops, emulsion, dry powder, suspension, pastille, patch, suppository or syrup.

13. A method as claimed in claim 2, wherein the active ingredient is administered to the mucous membranes of the nasal cavity.

14. A method as claimed in claim 2, wherein the active ingredient is administered as a solution or suspension spray or as a powder.

15. A method as claimed in any of claim 2, in which the active ingredient is administered between 1 minute and 2 hours prior to sleeptime.

16. A pharmaceutical formulation for the treatment or prevention of grogginess, drowsiness or lethargy on waking after sleeping, comprising triprolidine or a salt or hydrate thereof as active ingredient in association with a pharmaceutically acceptable carrier therefor and instructions for administration thereof at or just before the desired sleeping time.

17. A pharmaceutical formulation for enabling an individual to wake more refreshed after sleeping, comprising triprolidine or a salt or hydrate thereof as active ingredient in association with a pharmaceutically acceptable carrier therefor and instructions for administration thereof at or just before the desired sleeping time.

18. The pharmaceutical formulation as claimed in claims 17, wherein the instructions for administration instruct a single dose of the active ingredient of triprolidine of less than 5 mg prior to sleeptime.

19. The pharmaceutical formulation as claimed in claim 17, wherein the instructions for administration instruct a single dose of the active ingredient of triprolidine of between 0.01 and 4.9 mg prior to sleeptime.

20. A waking refreshed aid comprising triprolidine or a salt or hydrate thereof as active ingredient in association with a pharmaceutically acceptable carrier therefor and instructions for administration thereof at or just before the desired sleeping time.

21. A waking refreshed aid as claimed in claim 20, wherein the instructions for administration instruct a single dose of the active ingredient of less than 5 mg prior to sleeptime.

22. A waking refreshed aid as claimed in claim 20, wherein the instructions for administration instruct a single dose of the active ingredient of triprolidine of between 0.01 and 4.9 mg prior to sleeptime.

23. The use of triprolidine or a salt or hydrate thereof as active ingredient in the preparation of a composition for enabling an individual to wake refreshed after sleeping.

24. The use of triprolidine or a salt or hydrate thereof as active ingredient in the preparation of a composition for enabling an individual to wake refreshed after sleeping.

25. The use of triprolidine or a salt or hydrate thereof as active ingredient in the preparation of a medicament for enabling an individual to wake refreshed after sleeping.

26. The use of triprolidine or a salt or hydrate thereof in the preparation of a sleep aid which also enables an individual to wake refreshed after sleeping.

27. The use of triprolidine or a salt or hydrate thereof as active ingredient of a sleep aid which also enables an individual to wake refreshed after sleeping.

28. The use of triprolidine or a salt or hydrate thereof as active ingredient in the preparation of a medicament for the treatment or prevention of a sleep disorder which also enables an individual to wake refreshed after sleeping.

29. The use as claimed in any of claim 23 , wherein the dose of triprolidine administered to the user prior to sleeptime is between 0.01 mg and 4.9 mg.

30. The use as claimed in any of claim 23. wherein the dose of triprolidine administered to the user before sleeptime is less than 5 mg.

31. Use as claimed in any of claim 23, wherein the triprolidine is in the form of triprolidine hydrochloride.

32. Use as claimed in any one of claim 23, wherein the composition is for oral administration.

33. Use as claimed in any of claim 23, wherein the composition is in the form of a tablet, capsule, drink, lozenge, drops, emulsion, dry powder, suspension, pastille, patch, suppository or syrup.

34. Use as claimed in any one of claim 23, wherein the composition is for administration to the mucous membranes of the nasal cavity.

35. Use as claimed in claim 34, wherein the composition is a solution or suspension or a powder.

36. The use as claimed in claim 23, wherein the triprolidine forms the active ingredient of a formulation which contains a blend of two or more diluents, one of which may also serve as a disintegrant.

37. The use as claimed in claim 23. wherein the triprolidine forms the active ingredient of a formulation, which comprises a saccharide diluent.

38. The use as claimed in claim 23, wherein the triprolidine formulation further comprises a disintegrant.

39. The use as claimed in claim 23, wherein the triprolidine formulation further comprises the saccharide diluent and the disintegrant in the ratio of 1-10 parts by weight saccharide diluent to 1 part by weight of disintegrant.

40. The use as claimed in claim 37, wherein the saccharide diluent is lactose, and the disintegrant is croscarmellose sodium.

41. The use as claimed in claim 23, wherein the triprolidine formulation further comprises a lubricant.

42. The use as claimed in claim 41, wherein the lubricant is magnesium stearate.

43. The use as claimed in claim 23, wherein the triprolidine formulation is formed with a coating of a hydrophilic polymer.

44. The use as claimed in claim 43, wherein the hydrophilic polymer is a methylated cellulose derivative.

45. The use as claimed in claim 23, which is free of ingredients intended or effective to sustain or prolong release of the active ingredient.

46. A method of manufacturing a formulation as claimed in claim 23, which involves direct compression of the ingredients into a tablet without an intermediate granulation stage.
